(19) United States
(12) Plant Patent Application Publication
(10) Pub. No.: US 2017/0142887 P1
(43) Pub. Date: May 18, 2017

(54) Vanda plant named 'SPCDW1515'

(50) ;

(71) Applicant: Suphatchatwong Innovation Co., Ltd., Chonburi (TH)

(72) Inventor: Thumrong Suphachadiwong, Bangphra (TH)

(73) Assignee: Suphatchatwong Innovation Co., Ltd., Chonburi (TH)

(21) Appl. No.: 15/330,348

(22) Filed: Sep. 9, 2016

(30) Foreign Application Priority Data
Sep. 15, 2015 (QZ) 2015/2164

(51) Int. Cl. A01H 5/02

(52) U.S. Cl. PLT/311

(57) ABSTRACT

A new and distinct Vanda cultivar named ‘SPCDW1515’ which is characterized by sepals and petals which are white to purple-violet and blotched violet, sepals and petals which are heavily dotted and marbled violet to violet-blue, a labellum with lateral lobes which are white and fading to yellow and a violet to violet-blue central lobe, as well as the stability of these characteristics from generation to generation.

### CROSS REFERENCE TO RELATED APPLICATIONS

This application claims priority to the Community Plant Variety Rights application number 2015/2164, filed Sep. 15, 2015, which is herein incorporated by reference.

Latin name of the genus and species: The Latin name of the genus and species of the novel variety disclosed herein is Vanda Jones ex R. Br..

Variety denomination: The inventive variety of Vanda disclosed herein has been given the variety denomination ‘SPCDW1515’.

### BACKGROUND OF THE INVENTION

Parentage: The Vanda variety ‘SPCDW1515’ is the result of a planned breeding program. The new variety originated as a seedling from the crossing of an unnamed Vanda seed parent (not patented) with an unnamed Vanda pollen parent (not patented). Both parents were developed by, and are owned by, the inventor and were never commercially released. The crossing was made by the inventor in his research greenhouse in Bangphra, Thailand in September of 2004.

The new variety was first selected in December of 2009, in a research greenhouse in Bangphra, Thailand belonging to the inventor. After identifying the new variety as a potentially interesting selection, the inventor continued confidential testing and propagation of ‘SPCDW1515’, assessing stability of the unique characteristics of this variety.

Asexual Reproduction: Asexual reproduction of the new cultivar ‘SPCDW1515’, by way of mericloning, was first initiated in October of 2010 at a commercial laboratory belonging to the inventor in Bangphra, Thailand. Access to all plants was restricted, as plants were kept in a greenhouse not open to the public, and tissue culture plantlets were in a laboratory belonging to the inventor, and not accessible to the public. Through six subsequent generations, the unique features of this cultivar have proven to be stable and true to type.

### SUMMARY OF THE INVENTION

The cultivar ‘SPCDW1515’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘SPCDW1515’. These characteristics in combination distinguish ‘SPCDW1515’ as a new and distinct Vanda cultivar:
1. Vanda ‘SPCDW1515’ exhibits sepals and petals which are white to purple-violet and blotched violet; and
2. Vanda ‘SPCDW1515’ exhibits sepals and petals which are heavily dotted and marbled violet to violet-blue; and
3. Vanda ‘SPCDW1515’ exhibits a labellum with lateral lobes which are white and fading to yellow, and a violet to violet-blue central lobe.

### BRIEF DESCRIPTION OF THE FIGURES

FIG. 1 illustrates, as nearly true as it is reasonably possible to make the same in color photographs of this type, an exemplary plant of ‘SPCDW1515’ grown in a commercial greenhouse in Honselersdijk, the Netherlands. This plant is approximately 6 years old from the time of mericloning, shown planted in an 8 by 8 cm container.

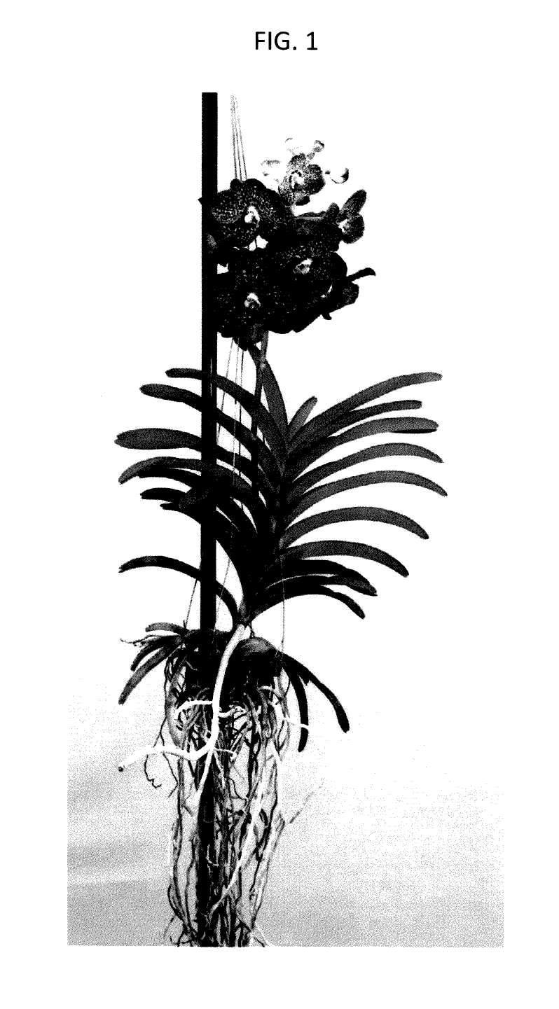

FIG. 2 illustrates, as nearly true as it is reasonably possible to make the same in color photographs of this type, the typical foliage of ‘SPCDW1515’.

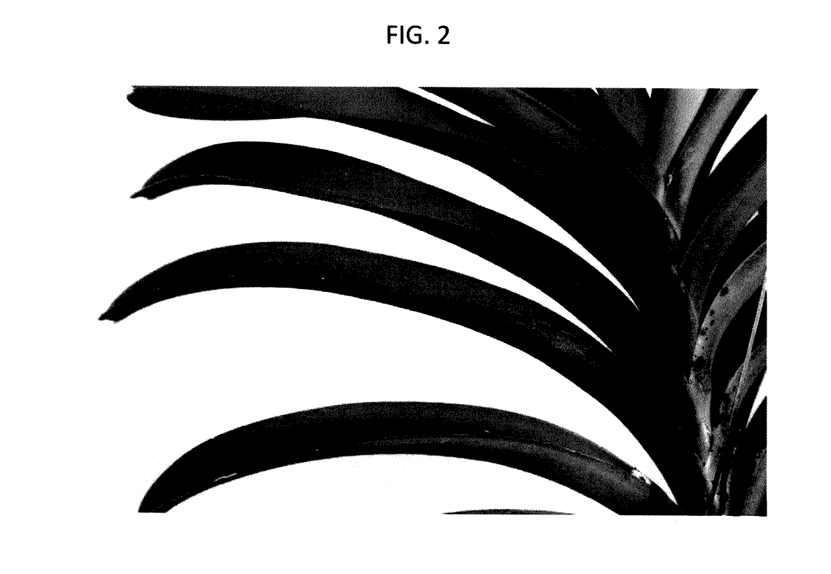

FIG. 3 illustrates, as nearly true as it is reasonably possible to make the same in color photographs of this type, the typical inflorescence of ‘SPCDW1515’.

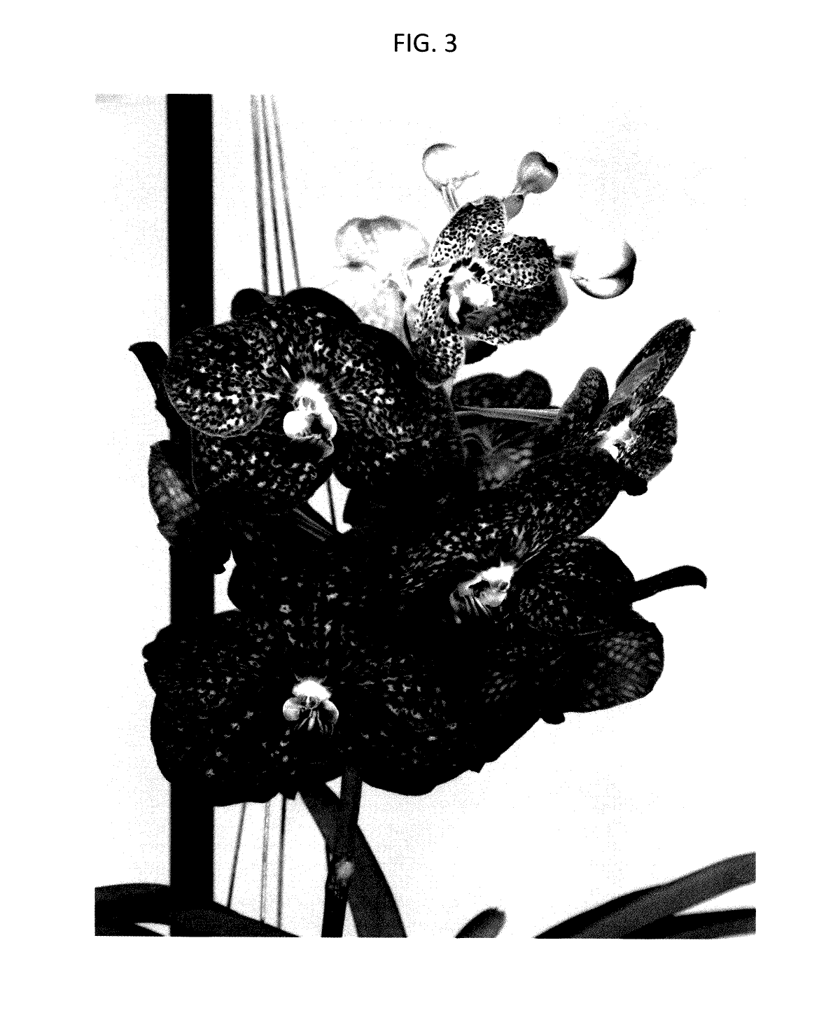

FIG. 4 illustrates, as nearly true as it is reasonably possible to make the same in color photographs of this type, the typical flower of ‘SPCDW1515’.

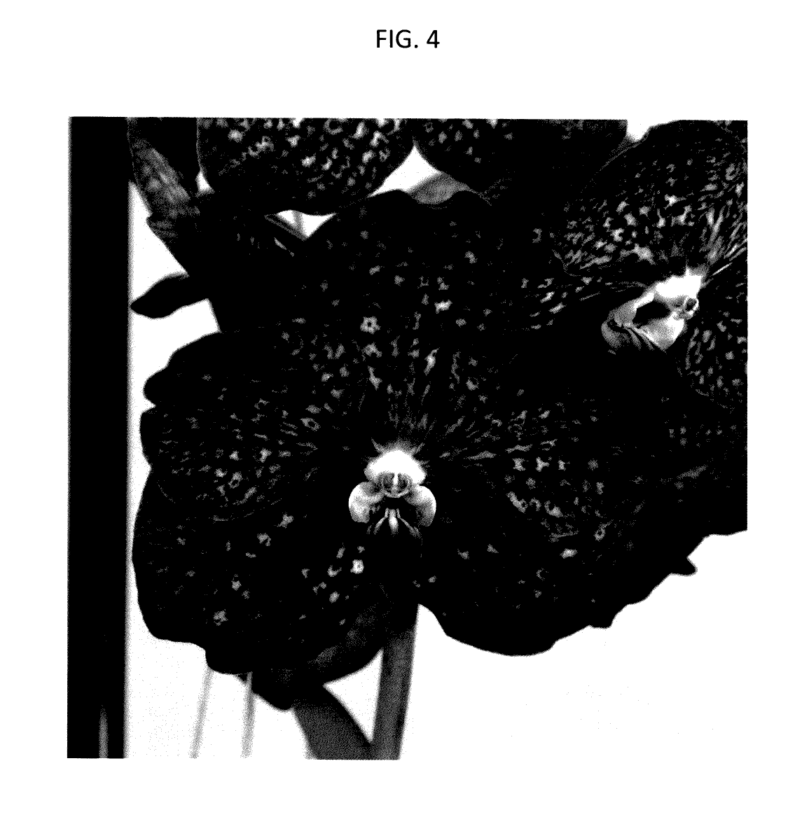

### BOTANICAL DESCRIPTION OF THE PLANT

The following observations and measurements made in August of 2016 describe averages from a sample set of six specimens of six year-old ‘SPCDW1515’ plants grown in a greenhouse in Honselersdijk, the Netherlands. Temperatures were approximately 23 degrees Celsius during the day and 19 degrees Celsius at night. No photoperiodic or chemical treatments were given to the plants. No artificial light was given to the plants.

Those skilled in the art will appreciate that certain characteristics will vary with older or, conversely, with younger plants. ‘SPCDW1515’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climatic and cultural conditions. Color notations are based on The Royal Horticultural Society Colour Chart, The Royal Horticultural Society, London, 2015 (sixth edition).

A botanical description of ‘SPCDW1515’ and comparisons with other varieties of Vanda are provided below.
Plant description:
   Growth habit.—Perennial; monopodial, upright. Single monopodial leafy stem, with flowers carried slightly above the leaf plane with inflorescences growing from the leaf axils.
   Average height from base (plant grown in hanging basket, so no soil line present).—to foliar plane 36.0 cm.
   Average height from base (plant grown in hanging basket, so no soil line present).—to top of floral plane 55.5 cm.
   Plant spread.—Average of 35.7 cm.
   Appropriate container size.—8 cm by 8 cm rectangular hanging basket.
   Growth rate.—Moderate.
   Plant vigor.—Moderate.
   Propagation type.—Tissue culture propagation using the base of the leaves.
   Propagation details.—The time needed from for the start of a mericlone to get the small plants ready for deflasking is approximately 24 months under a controlled temperature of about 26 degrees Celsius. After that period, plants have matured roots. Plants start to make roots in the last 6 months in flasks. After deflasking they remain in a shaded and controlled environment.
   Disease and pest resistance or susceptibility.—Similar to other Vanda varieties.
   Environmental tolerances.—Adapt to, at least, USDA Zones 11 and 12 and temperatures as high as 40 degrees Celsius; high tolerance to rain; moderate to high tolerance to wind.
Root system:
   General.—Densely rooting; with aerial roots only.
   Dimensions, mature roots.—56.9 cm long with a diameter of 0.4 cm, on average.
   Texture.—Very fleshy; non-fibrous.
   Color.—Greyed-Green, near RHS 192A to 192B; root apex is yellow-green, RHS 147A to 147B.
Stem:
   Branching habit.—Monopodial; no lateral branches present.
   Number of primary (main) stems per plant.—One.
   Number of secondary (lateral) branches per plant.—None.
   Appearance and shape.—Circular, formed by the leaf sheaths.
   Length.—24.0 cm.
   Diameter.—1.1 cm.
   Internode length.—1.0 cm.
   Luster.—Matte.
   Aspect.—Near vertical.
   Strength.—Very strong.
   Color, juvenile.—Yellow-green, in between near RHS 150C and 150D.
   Color, mature.—Yellow-green, in between near RHS 150C and 150D.
   Color at internodes.—Yellow-green, in between near RHS 150C and 150D.
   Pubescence.—Glabrous.
Foliage:
   Arrangement.—Distichous.
   Dimensions.—18.4 cm long and 2.3 cm wide, on average.
   Shape of blade.—Ligulate; strongly carinate.
   Aspect.—Moderately curved.
   Apex.—Unequal praemorse.
   Base.—Sheathing.
   Sheath dimensions.—3.0 cm long and 1.2 cm wide, on average.
   Sheath color.—Yellow-green, near RHS 145B and 145C.
   Margin.—Entire.
   Pubescence, texture and luster of adaxial surface.—Glabrous, smooth, and very slightly glossy.
   Pubescence, texture and luster of abaxial surface.—Glabrous, smooth, and matte.
   Color.—Juvenile foliage, adaxial surface — Green, near RHS 143B to 143C, and darker towards the margins, near NN 137B. Juvenile foliage, abaxial surface — Green, near RHS 143B to 143C. Mature foliage, adaxial surface — Green, near RHS 143A. Mature foliage, abaxial surface — In between green, RHS 143C, and yellow-green, RHS 144A.
   Venation.—Parallel.
   Venation color, adaxial surface.—Green, near RHS 143A.
   Venation color, abaxial surface.—In between green, RHS 143C, and yellow-green, RHS 144A.
   Petiole.—No petioles present; leaves sessile.
Inflorescence:
   Inflorescence arrangement.—Axially placed raceme.
   Natural flowering season.—Autumn into winter.
   Time to flower.—Approximately 10 months.
   Flowers per inflorescence.—Average of 13; 10 open flowers and 3 buds.
   Inflorescence height.—Approximately 21.6 cm (measured from base of lowest flower to top of upper flower).
   Inflorescence diameter.—Approximately 17.6 cm.
   Peduncle.—Dimensions — Approximately 40.2 cm long and 0.7 cm wide. Aspect — Generally at a 30 degree angle to lateral branch axis, varying between 20 and 40 degrees. Strength — Moderately strong. Texture and luster — Glabrous and moderately glossy. Color — Green, near RHS 143B to 143C.
Bud:
   Length.—Average of 1.7 cm.
   Diameter.—Average horizontal diameter is 1.6 cm; the vertical diameter is 2.0 cm.
   Shape.—Cubic, with a deltoid apex which is pointed downward.
   Pubescence, texture and luster.—Glabrous, smooth and matte.
   Color.—Yellow-green, near RHS 145D; apex is purple, near RHS 79D.
Flower:
   Flower type.—Zygomorphic (orchidaceous), typical shape for the genus Vanda.
   Flowering habit.—Moderate.
   Shape.—Zygomorphic, single; consisting of three sepals and three petals, one of which is transformed into a labellum.
   Aspect.—Flowers are outward-facing to slightly nodding.
   Vertical height.—Approximately 10.2 cm.
   Diameter.—Approximately 9.8 cm.
   Depth.—Approximately 3.5 cm.
   Fragrance.—No fragrance.
   Lastingness.—3 weeks, on average.
   Persistent.—Self-cleaning.
   Petals.—Quantity of Petals — Three unfused petals; two conventional petals and one labellum. Arrangement — One lateral petal on each side. Length — Approximately 4.6 cm. Width — Approximately 4.5 cm. Shape — Near orbicular. Apex — Obtuse. Base — Cuneate. Margin — Entire; slightly undulated. Pubescence, texture and luster of upper surface — Glabrous, velvety and matte. Pubescence, texture and luster of lower surface — Glabrous, velvety and matte. Color when opening, upper surface — White, near RHS NN155D, and blotched violet, near RHS N87C to N87D; moderately to heavily dotted violet to violet-blue, in between RHS 83A and N92A.
   Color when opening, lower surface.—White, near RHS NN155A, and tinged violet-blue particularly towards the apex and margins, near RHS 90D and lighter.
   Color when fully opened, upper surface.—Purple-violet, near RHS N82C and N82D, and blotched violet, near RHS 86B and N87A; heavily dotted violet to violet-blue, in between RHS 83A and N92A.
   Color when fully opened, lower surface.—Violet, near RHS 85C and 85D, and fading darker towards the top, near RHS N88C.
   Fading, upper surface.—Not fading.
   Fading, lower surface.—Not fading.
   Venation.—Densely reticulate.
   Venation color, fully opened, upper surface.—Purple-violet, near RHS N82C and N82D, and blotched violet, near RHS 86B and N87A.
   Venation color, fully opened, lower surface.—Violet, near RHS N88B.
Labellum:
   Shape.—Three-lobed, lateral lobes are involute and the central lobe is revolute.
   Length.—Central lobe — Approximately 2.0 cm. Lateral lobes — Approximately 0.7 cm.
   Width.—Central lobe — Approximately 1.1 cm. Lateral lobes — Approximately 0.7 cm.
   Apex, central lobe.—Emarginate.
   Apex, lateral lobes.—Obtuse.
   Base, central and lateral lobes.—Cuneate.
   Margin, central lobe.—Entire; revolute and slightly undulated.
   Margin, lateral lobes.—Entire; not undulate.
   Texture and luster of upper surface.—Central lobe — Glabrous, velvety, and matte. Lateral lobes — Glabrous, velvety, and matte.
   Texture and luster of lower surface.—Central lobe — Glabrous, velvety, and very slightly glossy. Lateral lobes — Glabrous, velvety, and matte.
   Color when opening, upper surface.—Central lobe — Violet, near RHS 86C to 86D. Lateral lobes — White, near RHS 155A, and fading to yellow, RHS 11B, towards the base.
   Color when opening, lower surface.—Central lobe — Violet, near RHS 86D, and fading to white, near RHS 155C, towards the base. Lateral lobes — White, near RHS 155B.
   Color when fully opened, upper surface.—Central lobe — Violet, near RHS 86A, and fading to violet-blue at the apex, RHS N89B. Lateral lobes — White, near RHS 155A, and fading to yellow, RHS 6C, towards the base; tinged purple-violet towards the apex, RHS N82D.
   Color when fully opened, lower surface.—Central lobe — Violet-blue, near RHS N92B, and fading lighter towards the base, near RHS 91C. Lateral lobes — White, near RHS 155B, and tinged violet at the apex, RHS 86D.
   Fading, upper surface.—Not fading.
   Fading, lower surface.—Not fading.
   Venation.—Densely reticulate.
   Venation color, fully opened, upper surface.—Central lobe — Violet, near RHS 86A, and fading to violet-blue at the apex, RHS N89B. Lateral lobes — White, near RHS 155A, and fading to yellow, RHS 6C, towards the base; tinged purple-violet towards the apex, RHS N82D.
   Venation color, fully opened, lower surface.—Central lobe — Violet-blue, near RHS N92B, and fading lighter towards the base, near RHS 91C.
   Lateral lobes — White, near RHS 155B, and tinged violet at the apex, RHS 86D.
   Callosities.—Dimensions — 1.5 cm long and 0.8 cm wide, on average. Color — White, near RHS N155A.
   Spurs.—Quantity — Average of 1. Length — Approximately 0.6 cm. Diameter — Approximately 0.4 cm. Vertical height — Approximately 0.5 cm, at the base. Shape — Short triangular. Apex — Bluntly acute. Base — Broad Cuneate. Pubescence, texture and luster — Glabrous, smooth, and matte. Color — Violet, near RHS 85D, and very slightly striped darker, near RHS 85B to 85C.
   Sepals.—Quantity — Average of 3 unfused sepals. Arrangement — One dorsal sepal and two lateral sepals. Length — Dorsal sepal — Approximately 4.9 cm. Lateral sepals — Approximately 5.0 cm. Width — Dorsal sepal — Approximately 4.7 cm. Lateral sepals — Approximately 5.6 cm. Shape — Dorsal sepal — Broad ovate. Lateral sepals — Near orbicular. Apex, dorsal and lateral sepals — Obtuse. Base, dorsal and lateral sepals — Cuneate. Margin, dorsal and lateral sepals — Entire; dorsal sepal are non-undulated and lateral sepals are slightly undulated. Pubescence, texture and luster — Glabrous, velvety, and matte on both the upper and lower sepal surfaces. Color when opening, upper surface — Dorsal sepal — White, near RHS NN155D, and blotched violet, near RHS N87C and N87D; sepals are moderately to heavily dotted violet to violet-blue, in between RHS 83A and N92A. Lateral sepals — White, near RHS NN155D, and blotched violet, near RHS 86C and 86D; sepals are moderately to heavily dotted violet to violet-blue, in between RHS 83A and N92A. Color when opening, lower surface — Dorsal sepal — Violet, near RHS 85C to 85D, and fading darker towards the apex, near RHS 86B to 86C. Lateral sepals — Violet, near RHS 85C to 85D, and fading darker towards the apex, near RHS 86B to 86C. Color when fully opened, upper surface — Dorsal sepal — White, near RHS NN155A, and blotched violet, RHS N88B; sepals are heavily dotted and marbled darker, near RHS 86A. Lateral sepals — White, near RHS NN155A, and blotched violet, RHS N88B; sepals are heavily dotted and marbled darker, near RHS 86A. Color when fully opened, lower surface — Dorsal sepal — Violet, near 85C and 85D, and fading darker towards the apex, near RHS N88C. Lateral sepals — Violet, near 85C and 85D, and fading darker towards the apex, near RHS N88C. Fading, upper surface — Not fading. Fading, lower surface — Not fading. Venation — Densely reticulate. Venation color, fully opened, upper surface — Dorsal sepal — White, near RHS NN155A, and blotched violet, RHS N88B; sepals are heavily dotted and marbled darker, near RHS 86A. Lateral sepals — White, near RHS NN155A, and blotched violet, RHS N88B; sepals are heavily dotted and marbled darker, near RHS 86A. Venation color, fully opened, lower surface — Dorsal sepal — Violet-blue, near RHS 90D, and fading violet towards the apex, in between near N88B and N88C. Lateral sepals — Violet-blue, near RHS 90D, and fading violet towards the apex, in between near N88B and N88C.
   Pedicels.—Length — Approximately 5.4 cm. Diameter — Approximately 0.5 cm. Angle — Approximately 75 degree angle to peduncle axis. Strength — Moderate. Texture — Glabrous, axially angled with 5 “ribs”, axially twisted (due to resupination). Luster — Glossy. Color — Purple-violet, near RHS N82B.
   Bract.—Quantity — One bract present at the base of each pedicel. Shape — Broad ovate; folded around the base of each pedicel. Dimensions — Average of 1.3 cm long and 1.3 cm wide. Apex — Broad acute. Base — Broad cuneate. Margins — Entire. Color, both surfaces — Yellow-green, near RHS N144B.
Reproductive organs:
   Column.—Length — Approximately 0.5 cm. Diameter — Approximately 0.6 cm. Color — White, NN155A, and tinged purple-violet, N82B.
   Ovary.—Position — Inferior Diameter — Approximately 0.3 cm. Color — Yellow-green, RHS 150C.
   Pollinia color.—Yellow-orange, RHS 14A.
Seed and fruit: No fruit and seed production has been detected to date.
Comparison with the parent plants: Plants of the new cultivar ‘SPCDW1515’ differ from the unnamed Vanda seed parent (not patented) by the characteristics described in Chart 1.

CHART 1

| Characteristic | ‘SPCDW1515’ | Unnamed seed parent |
|---|---|---|
| Flower color. | Background color is a | Background color is a |
| | darker violet-blue. | lighter blue. |
| Flower color | Heavily dotted. | Lightly dotted. |
| pattern. | | |
| Inflorescence. | More flowers on each | Fewer flowers on each |
| | raceme compared to the | raceme compared to the |
| | seed parent. | ‘SPCDW1515’. |
| Flower size. | Smaller than the seed | Larger than ‘SPCDW1515’. |
| | parent. | |
| Plant size. | More compact than the | Larger than ‘SPCDW1515’. |
| | seed parent. | |
| Plant vigor. | More vigorous; easier to | Less vigorous; more difficult |
| | cultivate and quicker to | to cultivate and slower to |
| | flower when compared to | flower when compared to |
| | the seed parent. | ‘SPCDW1515’. |

Plants of the new cultivar ‘SPCDW1515’ differ from the unnamed Vanda pollen parent (not patented) by the characteristics described in Chart 2.

CHART 2

| Characteristic | ‘SPCDW1515’ | Unnamed pollen parent |
|---|---|---|
| Flower color. | Non-uniform violet-blue. | Uniform dark violet-blue. |
| Inflorescence. | Fewer flowers on each | More flowers on each |
| | raceme compared to the | raceme compared to the |
| | seed parent. | ‘SPCDW1515’. |
| Flower size. | Larger than the pollen | Smaller than |
| | parent. | ‘SPCDW1515’. |
| Plant size. | Larger than the pollen | Smaller than |
| | parent. | ‘SPCDW1515’. |

Comparison with the most similar Vanda cultivar known to the inventor: Plants of the new cultivar ‘SPCDW1515’ are most similar to the cultivar, Vanda ‘Princess Mikasa’ (not patented in the United States). A comparison of ‘SPCDW1515’ with Vanda ‘Princess Mikasa’ is described in Chart 3.

CHART 3

| Characteristic | ‘SPCDW1515’ | ‘Princess Mikasa’ |
|---|---|---|
| Leaf length. | 18.4 cm. | 22.2 cm. |
| Flower, vertical | 10.2 cm. | 9.3 cm. |
| height. | | |
| Flower, | 9.8 cm. | 9.2 cm. |
| diameter. | | |
| Petal color; | White, near RHS | Violet, near RHS N88C, |
| upper surface, | NN155D, and blotched | and veined darker, RHS |
| when opening. | violet, near RHS N87C to | 86B to 86C; lower part of |
| | N87D; moderately to | petal finely dotted violet, |
| | heavily dotted violet to | near RHS 86A to 86B. |
| | violet-blue, in between | |
| | RHS 83A and N92A. | |
| Petal color; | White, near RHS | Violet, near RHS N88C, |
| lower surface, | NN155A, and tinged | and veined darker, RHS |
| when opening. | violet-blue particularly | 86B to 86C. |
| | towards the apex and | |
| | margins, near RHS 90D | |
| | and lighter. | |
| Petal color; | Purple-violet, near RHS | Violet, near RHS N87C. |
| upper surface, | N82C and N82D, and | |
| when fully | blotched violet, near RHS | |
| open. | 86B and N87A; heavily | |
| | dotted violet to violet-blue, | |
| | in between RHS 83A and | |
| | N92A. | |
| Petal color; | Violet, lighter than near | Violet, near RHS 86C. |
| lower surface, | RHS N87D, and fading | |
| when fully | darker, near RHS N87C. | |
| open. | | |
| Venation color; | Purple-violet, near RHS | Violet, in between near |
| upper petal | N82C and N82D, and | RHS 86A and N88A; |
| surface, when | blotched violet, near RHS | slightly closer to N88A. |
| fully open. | 86B and N87A. | |
| Venation color; | Violet, near RHS N88B. | Violet, near RHS 86A. |
| lower petal | | |
| surface, when | | |
| fully open. | | |
| Labellum apex. | Central lobe is | Central lobe is strongly |
| | emarginate, and lateral | emarginate, and lateral |
| | lobes are obtuse. | lobes are emarginate. |
| Central lobe of | Violet, near RHS 86A, and | Violet-blue, in between near |
| labellum; color | fading to violet-blue at the | RHS N92A and N92C; base |
| of the upper | apex, RHS N89B. | slightly lighter, near RHS |
| surface when | | 86A. |
| fully open. | | |
| Central lobe of | Violet-blue, near RHS | Violet, near RHS 86A, and |
| labellum; color | N92B, and fading lighter | fading lighter towards the |
| of the lower | towards the base, near | base, near RHS 86D. |
| surface when | RHS 91C. | |
| fully open. | | |
| Dorsal sepal | 4.9 cm long and 4.7 cm | 4.6 cm long and 4.0 cm |
| dimensions. | wide. | wide. |
| Lateral sepal | 5.0 cm long and 5.6 cm | 4.9 cm long and 4.6 cm |
| dimensions. | wide. | wide. |
| Sepal color; | Dorsal sepal is white, near | Lateral sepals and dorsal |
| upper surface, | RHS NN155D, and | sepal are violet, near N88C, |
| when opening. | blotched violet, near RHS | and veined darker, RHS |
| | N87C and N87D; | 86B to 86C. |
| | moderately to heavily | |
| | dotted violet to violet-blue, | |
| | in between RHS 83A and | |
| | N92A. Lateral sepals are | |
| | white, near RHS NN155D, | |
| | and blotched violet, near | |
| | RHS 86C and 86D; | |
| | moderately to heavily | |
| | dotted violet to violet-blue, | |
| | in between RHS 83A and | |
| | N92A. | |
| Sepal color; | Dorsal sepal is violet, near | Lateral sepals and dorsal |
| lower surface, | RHS 85C to 85D, and | sepal are violet, near N88C, |
| when opening. | fading darker towards the | and veined darker, RHS |
| | apex, near RHS 86B to | 86B to 86C. |
| | 86C. Lateral sepals are | |
| | violet, near RHS 85C to | |
| | 85D, and fading darker | |
| | towards the apex, near | |
| | RHS 86B to 86C. | |
| Sepal color; | Dorsal sepal is white, near | Dorsal sepal is violet, near |
| upper surface, | RHS NN155A, and | RHS N87C. Lateral sepals |
| when fully | blotched violet, RHS | are violet, in between near |
| open. | N88B; heavily dotted and | RHS N87C to N87D. |
| | marbled darker, near RHS | |
| | 86A. Lateral sepals are | |
| | white, near RHS NN155A, | |
| | and blotched violet, RHS | |
| | N88B; heavily dotted and | |
| | marbled darker, near RHS | |
| | 86A. | |
| Sepal color; | Dorsal sepal is violet, near | Dorsal sepal is violet, in |
| lower surface, | 85C and 85D, and fading | between near RHS N87B |
| when fully | darker towards the apex, | and N87C. Lateral sepals |
| open. | near RHS N88C. Lateral | are violet, near RHS N87C. |
| | sepals are violet, near | |
| | 85C and 85D, and fading | |
| | darker towards the apex, | |
| | near RHS | N88C. |
| Venation color; | Dorsal sepal is white, near | Dorsal sepal is violet, in |
| upper sepal | RHS NN155A, and | between near RHS 86A and |
| surface, when | blotched violet, RHS | N87A. Lateral sepals are |
| fully open. | N88B; heavily dotted and | violet, near RHS 86A. |
| | marbled darker, near RHS | |
| | 86A. Lateral sepals are | |
| | white, near RHS NN155A, | |
| | and blotched violet, RHS | |
| | N88B; heavily dotted and | |
| | marbled darker, near RHS | |
| | 86A. | |
| Venation color; | Dorsal sepal is violet-blue, | Dorsal sepal is violet, in |
| lower sepal | near RHS 90D, and fading | between near RHS 86A and |
| surface, when | violet towards the apex, in | 86B. Lateral sepals are |
| fully open. | between near N88B and | violet, in between near RHS |
| | N88C. Lateral sepals are | 86A and N88A. |
| | violet-blue, near RHS | |
| | 90D, and fading violet | |
| | towards the apex, in | |
| | between near N88B and | |
| | N88C. | |
| Spur | 0.6 cm long, 0.4 cm in | 0.8 cm long, 0.25 cm in |
| dimensions. | diameter, and 0.5 cm in | diameter, and 0.5 cm in |
| | vertical height. | vertical height. |
| Spur shape. | Short triangular. | Flattened narrow triangular. |
| Spur color. | Violet, near RHS 85D, | Violet, near RHS 85B to |
| | and very slightly striped | 85C, and striped darker, |
| | darker, near RHS 85B to | near RHS N87C to N87D; |
| | 85C. | apex is darker, near RHS |
| | | 86B to 86C. |

That which is claimed is:

1. A new and distinct variety of Vanda plant named ‘SPCDW1515’, substantially as described and illustrated herein.
